(19) United States
(12) Patent Application Publication
(10) Pub. No.: US 2020/0273544 A1
(43) Pub. Date: Aug. 27, 2020

(54) METHOD FOR DETERMINING PROTONATION STATES OF PROTEIN ON BASIS OF CONSTANT-PH MOLECULAR DYNAMICS SIMULATION

(71) Applicant: SHENZHEN JINGTAI TECHNOLOGY CO., LTD., Guangdong (CN)

(72) Inventors: Xiao WAN, Guangdong (CN); Fenglei CAO, Guangdong (CN); Mingjun YANG, Guangdong (CN); Jian MA, Guangdong (CN); Lipeng LAI, Guangdong (CN); Shuhao WEN, Guangdong (CN)

(73) Assignee: SHENZHEN JINGTAI TECHNOLOGY CO., LTD., Guangdong (CN)

(21) Appl. No.: 16/740,504

(22) Filed: Jan. 13, 2020

(30) Foreign Application Priority Data
Feb. 21, 2019 (CN) 201910129727.2

(51) Int. Cl. G16C 10/00

(52) U.S. Cl. CPC G16C 10/00

(57) ABSTRACT

A method for determining protonation states of a protein on the basis of constant-pH molecular dynamics simulation includes: calculating ΔG_{elec,ref} of a reference compound; setting reasonable initial protonation states according to a simulation target pH value; performing constant-pH molecular dynamics simulation, and restricting positions of protein main chain atoms; setting amino acid residues with a protonation state proportion of higher than 99% or lower than 1% to be not titrated and other amino acid residues to be titrated, performing conventional constant-pH molecular dynamics simulation; setting amino acid residues with a protonation state proportion of higher than 90% or lower than 10% to be not titrated and other amino acid residues to be titrated, performing constant-pH molecular dynamics simulation under conditions of pH−0.5, pH−0.2, pH, pH+0.2, pH+0.5, respectively; performing Hill equation fitting to obtain a final pKa, wherein the protonation state can be determined by pKa.

### CROSS-REFERENCE TO RELATED APPLICATION

This application claims the priority benefit of China application serial no. 201910129727.2, filed on Feb. 21, 2019. The entirety of the above-mentioned patent application is hereby incorporated by reference herein and made a part of this specification.

### BACKGROUND

### Technical Field

The present invention belongs to the field of molecular dynamics, and particularly relates to a method for determining protonation states of a protein on the basis of constant-pH molecular dynamics simulation.

### Description of Related Art

Molecular dynamics (MD) simulation is one of the important tools for studying the structure and function of proteins in the field of biomolecules. For a protein in an organism, its structure and function strongly depend on the pH environment in which it is located. This dependence is mainly caused by the change of the main protonation states of titratable amino acids with the change of pH (mainly the side chains of acidic and basic amino acids). The protonation states of these residues have a profound impact on the stability of the protein system, the interaction between the protein system and the surrounding environment, and the catalytic mechanism of acid-base catalysis or nucleophilic reactions depending on a specific protonation state.

At present, the determination of the protonation states mainly includes empirical methods such as H ++ or PROPKA, and dynamics simulation methods, such as constant-pH molecular dynamics simulation. The former is fast but relatively inaccurate, and the latter is relatively accurate but time consuming.

Constant-pH molecular dynamics simulation adds Monte Carlo Sampling (MC) on the basis of conventional molecular dynamics simulation to achieve conformation and momentum sampling from a set of fixed protonation state MD. The MC is used to sample the protonation states in fixed conformations throughout the entire MD process. In the MC sampling step, a titration residue and its new protonation state are randomly selected. The transition free energy of this protonation or deprotonation process can be calculated as follows:

ΔG=k_{B}(pH−pK_{a/ref})ln10+ΔG_{elec}ΔG_{elec/ref}

where k_{B} refers to Boltzmann constant, T refers to temperature, pH refers to the specified solution pH, pK_{a,ref} refers to the pKₐ of a suitable reference compound, ΔG_{elec} refers to the electrostatic term of the free energy calculated by the titration group in the protein, and ΔG_{elec,ref} refers to the electrostatic term of the protonation state transition free energy of the reference compound. A reference compound with a known pKₐ is introduced here for calculation.

Because in practice, the transient pKₐ of certain residues in a protein has a high correlation with their conformations, the transient pKₐ and the conformation affect each other. In the conventional constant-pH method, the conformational fluctuation range of a protein is larger than that of conventional MD. Therefore, the phenomenon that the protein is confined to the wrong protonation states and the relatively high energy conformation will appear in the simulation.

### SUMMARY

In order to better control the development direction of the simulation and improve the accuracy of the results, the present invention provides a method for accurately determining the protonation states of a protein on the basis of discrete constant-pH molecular dynamics simulation in AMBER.

Specific technical solutions are as follows:

A method for determining protonation states of a protein on the basis of constant-pH molecular dynamics simulation includes the following steps:

(1) ΔG_{elec,ref} of a reference compound is calculated.

AMBER defines a plurality of common amino acid residue reference compounds in a protein. If there are undefined ones in the simulation system, you need to define them yourself.

The thermodynamic integration method is used to calculate the free energy ΔG of the possible protonation state transition of each reference compound in a GB solvation model. The initial ΔG_{elec,ref} is given by ΔG-pKₐRTln10;

In order to give a more accurate ΔG_{elec,ref}, it needs to be corrected: According to the Henderson-Hasselbalch (HH) equation, when the pH is equal to pKₐ, the protonation proportion and the deprotonation proportion are equal. A simple conventional CpHMD simulation is performed, and the simulation parameter settings should be as consistent as possible with those of subsequent simulations. The simulation pH is set to be the reference compound pKₐ, and the correction is performed as follows according to derivation: ΔG_{elec,ref} (after correction)=ΔG_{elec,ref} (before correction)-1nK, where K refers to the ratio of the deprotonation states to the protonation states in the simulation: the ΔG_{elec,ref} after correction is used to simulate the reference compound under the condition of pH being pKₐ, and the ratio of the protonation states to the deprotonation states should be 1:1, otherwise the correction needs to be continued.

If the reference compound has multiple protonation states, each possible protonation state transition and its corresponding ΔG_{elec,ref} need to be defined.

(2) Reasonable initial protonation states are set based on the simulation target pH. The positions of all heavy atoms in the protein are restricted at the target pH, constant-pH molecular dynamics simulation is performed for a certain period of time, the proportion of protonation states of all titratable amino acids in the simulation is calculated statistically, and dominant protonation states are considered as the initial protonation states for the next step.

(3) Conventional constant-pH molecular dynamics simulation is performed for a certain period of time to restrict the positions of protein main chain atoms. The proportion of protonation states of all titrated amino acids in the simulation is calculated statistically; amino acid residues with a protonation state proportion of higher than 99% or lower than 1% are set to be not titrated, their protonation states are set to be large-proportion protonation transition, and other amino acid residues are set to be titrated, as the initial protonation states for next step.

(4) Conventional constant-pH molecular dynamics simulation is performed for a certain period of time. The proportion of protonation states of all titrated amino acids in the simulation is calculated statistically; amino acid residues with a protonation state proportion of higher than 90% or lower than 10% are set to be not titrated, other amino acid residues are set to be titrated, as the initial protonation states for next step.

(5) The constant-pH molecular dynamics simulation is performed under the conditions of pH−0.5, pH−0.2, pH, pH+0.2, and pH+0.5, respectively. The proportion of protonation states of all titrated amino acids changing with pH in the simulation is statistically calculated, and the final pKₐ is obtained by fitting the Hill equation. The protonation states may be determined by pKₐ.

The method for determining protonation states of a protein on the basis of constant-pH molecular dynamics simulation provided by the present invention has the following technical advantages:

(1) The correction method for calculating the ΔG_{elec,ref} of a reference compound is provided.

(2) For the case of a protein containing more titratable amino acids, the conventional constant-pH molecular dynamics simulation method is very time-consuming and slow in convergence. This method determines the protonation states through multiple steps in a step-by-step manner. Amino acids titrated subsequently become less and less, the calculation time is shorter and shorter, and the convergence is faster.

(3) There are more and more amino acids with determined protonation states, and fewer and fewer titrated amino acids, thus achieving more accurate results.

### DESCRIPTION OF THE EMBODIMENTS

The specific technical solution of the present invention will be described with reference to the embodiments.

Since it is difficult to determine the protonation states of amino acids in a protein experimentally, the benefits are explained below by the results of other studies in the reference documents.

The structure of a complex covalently bound by the neurotoxic agent sarin and acetylcholinesterase in combination with the small molecule antidote HI6 has a PDB number of 2WHP. A lot of documents have studied this structure in order to clarify the mechanism of HI6 detoxification and to design a more efficient antidote. The protein structure contains a total of 548 amino acids, and the amino acids titrated in the simulation include aspartic acid, glutamic acid, histidine, lysine, and tyrosine. A recent document (Driant, T.; Nachon, F.; Ollivier, C.; Renard, P. Y.; Derat, E., On the Influence of the Protonation States of Active Site Residues on AChE Reactivation: A QM/MM Approach Chembiochem 2017, 18 (7), 666-675.) pointed out that the detoxification reaction depends on the protonated glutamic acid 202 and histidine 447, while an oxime antidote should be deprotonated. Under the condition that the target pH is 7, the present embodiment cannot simulate the protonation states in the document through the conventional constant-pH molecular dynamics simulation; however, the results consistent with the results of the document are obtained through the improved constant-pH molecular dynamics simulation process.

TABLE 1

| | method 1 | method 2 |
|---|---|---|
| | pKa | pKa |
| GL4_202 | 6.28 | 7.71 |
| HIP_212 | 7.03 | 7.17 |
| LYS_332 | 9.17 | 8.94 |
| GL4_334 | 3.6 | 3.07 |
| HIP_405 | 5.44 | 5.23 |
| HIP_432 | 6.32 | 6.63 |
| HIP_447 | 3.39 | 5.89 |
| GL4_450 | 4.48 | 2.94 |
| GL4_452 | 6.03 | 6.57 |
| HI6_543 | 6.79 | 5.39 |

In Table 1 above, method 1 indicates the results of a conventional constant-pH molecular dynamics simulation, and method 2 indicates the process of the present invention. Other titratable amino acids not shown in the table above are in the conventional protonation state, that is, acidic amino acids are deprotonated and basic amino acids are protonated. It can be seen that the pKₐ calculation of method 2 is closer to the protonation states reported in the document.

What is claimed is:

1. A method for determining protonation states of a protein on the basis of constant-pH molecular dynamics simulation, comprising the following steps:
(1) calculating ΔG_{elec,ref} of a reference compound, wherein a thermodynamic integration method is used to calculate free energy AG of a possible protonation state transition of the reference compound in a GB solvation model, and then an initial ΔG_{elec,ref} is given by ΔG-pKₐRTln10; and
if the reference compound has multiple protonation states, each possible protonation state transition and its corresponding ΔG_{elec,ref} need to be defined;
(2) setting reasonable initial protonation states based on a simulation target pH, wherein positions of all heavy atoms in the protein are restricted at a target pH, constant-pH molecular dynamics simulation is performed for a certain period of time, a proportion of protonation states of all titratable amino acids in the constant-pH molecular dynamics simulation is calculated statistically, and dominant protonation states are considered as initial protonation states for next step;
(3) performing conventional constant-pH molecular dynamics simulation for a certain period of time to restrict positions of protein main chain atoms, statistically calculating a proportion of protonation states of all titrated amino acids in the conventional constant-pH molecular dynamics simulation; and setting amino acid residues with a protonation state proportion of higher than 99% or lower than 1% to be not titrated, their protonation states to be large-proportion protonation transition, and other amino acid residues to be titrated, as initial protonation states for next step;
(4) performing the conventional constant-pH molecular dynamics simulation for a certain period of time, statistically calculating a proportion of protonation states of all titrated amino acids in the conventional constant-pH molecular dynamics simulation; and setting amino acid residues with a protonation state proportion of higher than 90% or lower than 10% to be not titrated and other amino acid residues to be titrated, as initial protonation states for next step; and
(5) performing the constant-pH molecular dynamics simulation under conditions of pH−0.5, pH−0.2, pH, pH+0.2, pH+0.5, respectively; statistically calculating a proportion of protonation states of all titrated amino acids changing with pH in the constant-pH molecular dynamics simulation, and fitting a Hill equation to obtain a final pKₐ, wherein the protonation states are determined by pKₐ.

2. The method for determining protonation states of the protein on the basis of constant-pH molecular dynamics simulation according to claim 1, wherein the ΔG_{elec,ref} in step (1) is corrected as follows: according to a Henderson-Hasselbalch equation, when pH is equal to pKₐ, a protonation proportion and a deprotonation proportion are equal; conventional CpHMD simulation is performed, and simulation parameter settings are as consistent as possible with those of subsequent simulations; a simulation pH is set to be a reference compound pKₐ, and correction is performed as follows according to derivation:
ΔG_{elec,ref} (after correction)=ΔG_{elec,ref} (before correction)-lnK, where K refers to a ratio of deprotonation states to protonation states in the conventional CpHMD simulation;
the ΔG_{elec,ref} after correction is used to simulate the reference compound under the condition of pH being pKₐ, and the ratio of the protonation states to the deprotonation states is 1:1, otherwise the correction needs to be continued.
